(19) United States
(12) Patent Application Publication
(10) Pub. No.: US 2026/0251557 A1
(43) Pub. Date: Aug. 27, 2026

(54) Saliva Based System and Method for Detecting VOCs, General Health Issues, and Disease States

(71) Applicant: ORTHONU LLC, Rumson, NJ (US)

(72) Inventor: Sima Yakoby Epstein, Rumson, NJ (US)

(21) Appl. No.: 19/643,705

(22) Filed: Apr. 9, 2026

Related U.S. Application Data
(63) Continuation-in-part of application No. 17/557,695, filed on Dec. 21, 2021.
(63) Continuation-in-part of application No. 19/176,113, filed on Apr. 10, 2025.
(60) Provisional application No. 63/632,506, filed on Apr. 10, 2024.
(60) Provisional application No. 63/786,272, filed on Apr. 9, 2025.

(51) Int. Cl. G01N 21/29; G01N 21/77; G01N 33/487

(52) U.S. Cl. CPC G01N 21/293; G01N 33/487; G01N 2021/7759; G01N 2800/18; G01N 2800/52

(57) ABSTRACT

A method includes contacting one or more volatile organic compounds (“VOCs”) evolved from a sample to one or more detection regions configured for sensitivity against one or more VOCs. The contacting causes a change to the detection regions that is visible to the human eye. The method also includes determining the presence or absence of a VOC by comparing the change to the detection regions to a key, with the key being a visible change. A system utilizing the method is also described.

### CROSS-REFERENCE TO RELATED APPLICATIONS

This application claims priority to U.S. Provisional Patent Application No. 63/786,272, filed Apr. 9, 2025, U.S. Provisional Patent Application No. 63/632,506, filed Apr. 10, 2024, is a continuation in part of U.S. patent application Ser. No. 17/557,695, filed Dec. 21, 2021, and is a continuation in part of U.S. patent application Ser. No. 19/176,113, filed Apr. 10, 2025, the disclosures of which are incorporated herein by reference in their entirety.

### FIELD

The invention relates to the field of saliva and/or breath-based detection systems, including breath and/or saliva sensing for detecting halitosis and general health issues.

### BACKGROUND

Oral health is linked to overall health, with the mouth providing access to both the digestive and respiratory tracts. Existing methods for testing oral health are limited. In orthodontic and oral health care of patients, there are at least three areas of concern that must be addressed, and which are currently under-addressed in consumer solution products. These include 1) emergency care, 2) oral hygiene, and 3) visual challenges. Emergency care is needed to address discomfort, pain, sensitivity, ulcerations, dry mouth, and the like. Oral hygiene involves long term oral care and prevention. Oral hygiene includes maintenance care for preventing halitosis and preventing or treating gingivitis and the increase in gram negative bacteria on the tongue and periodontium that are associated with oral and orthodontia care. Avoiding gingivitis and reducing gram negative bacteria in a patient's mouth can prevent and/or deter bone loss and inflammation. This can help to maintain a healthy mouth, tongue, gums, and fresh breath. Visual challenges include the appearance of white decalcifications on teeth due to the start of cavities that can be attributed to an increase of gram-positive bacteria, among other challenges.

Gram negative bacteria lives on the tongue and inside the gum line around the periodontium. Gram negative bacteria is a main cause of halitosis in the mouth. Gram positive bacteria is found in the plaque accumulation of the pellicle that forms on the enamel of the tooth that then starts the decalcification process that can lead to cavities, also causing halitosis. Targeting these two types of bacteria with different ingredients and tools and differentiating them can solve many issues inherent in the changes in microbiome during orthodontic treatment.

In addition, overall health has been tied to health of the teeth and oral cavity. Oral health can offer clues to overall health. Problems in the mouth can affect the rest of the body. The mouth normally teems with bacteria, most of which is harmless. Some of the bacteria present in the mouth can cause disease or signal the existence of disease. Normally the body's natural defenses are improved by good oral health care, such as daily brushing and flossing. Good oral health care can keep bacteria under control. Without proper oral hygiene, bacterial can lead to oral infections, such as tooth decay and gum disease.

Saliva is an important component in the mouth because it washes away food and neutralizes acids produced by bacteria in the mouth. Saliva helps to protect against microbes that multiply and lead to disease. Certain common medications, such as decongestants, antihistamines, painkillers, diuretics and antidepressants, can reduce saliva flow.

Oral health can contribute to various diseases, such as endocarditis, cardiovascular disease, pregnancy and birth complications, and pneumonia, among others. For example, endocarditis, an infection of the inner lining of the heart chambers or valves, typically occurs when bacteria from another part of the body, such as the mouth, spreads through the bloodstream and attaches to certain areas in the heart. Some research suggests that heart disease, clogged arteries and stroke might be linked to the inflammation and infections that oral bacteria can cause. Periodontitis has been linked to premature birth and low birth weight. Certain bacteria in the mouth can be pulled into the lungs, which can cause pneumonia and other respiratory diseases.

Certain conditions may also affect overall oral health, such as diabetes, HIV/AIDS, osteoporosis, Alzheimer's disease, among others. Diabetes, for example, puts gums at greater risk by reducing the body's resistance to infection. Research shows that people with gum disease have a harder time controlling blood sugar levels. Oral problems, such as lesions are common with people having HIV/AIDS. Osteoporosis can cause periodontal bone loss and tooth loss. Worsening oral health is often seen in Alzheimer's disease progression.

Thus, oral health is important to overall health. Because of the lack of home care solutions, patients often simply live with the bad breath and plaque that builds up on their teeth. Patients are often unaware that their oral health has an impact on their overall health. On-the-go solutions and home-care solutions would assist patients in determining whether they need treatment to improve oral care.

There is also a need for an easy system for determining overall health based upon oral health. In particular, it is desirable to link gum and mouth health to other diseases by testing saliva. Such a system would permit a person to test their overall health at home. For example, cancer markers can be determined by testing saliva or breath. Other disease markers can also be identified from saliva and breath, even if unrelated to overall oral care. The present invention is directed toward a method for using saliva to determine both oral health and extra-oral health.

### SUMMARY

A saliva sensing device for detecting oral health and/or extraoral health includes a chemically treated test strip biomarker targeted to identify bacteria in the saliva. The test strip may be targeted to analyze one or more volatile organic compounds (“VOCs”), wherein the VOCs include one or more of ketones, alcohols, aldehyde, and sulfur. The test strip may measure unbound hormones in the saliva, with the measured unbound hormones being tied to one or more disease states. The test strip may measure lipidomic biomarkers. The test strip measures key lipids, and the measured key lipids are used in diagnosing diseases, diagnosing disease states, and developing treatment plans. The test strip may be targeted to test pathogens including one or more of S. mutans, S. sanguis, P. gingivalis, and F. nucleatum. The test strip may have a chemical treatment to detect gram negative bacteria. The test strip may have a chemical treatment to detect gram positive bacteria. The test strip may have a chemical treatment to detect one or more of the following:
1) Bacteria located in saliva due to low PH of saliva;
2) Bacteria located in saliva due to acidic saliva;
3) Bacteria production caused by insufficient hydration;
4) Bacteria production caused by insufficient salivary flow; and
5) Bacteria due to food related oral hygiene.

A breathalyzer for testing liquid particles from the breath for detecting bacteria in the mouth of a user includes a housing, a sensor coupled to the housing and extending outwardly therefrom for placing in the mouth of user and for collecting a saliva sample, and an analyzer for testing saliva that contacts the sensor. The analyzer may be a test strip sensor that collects saliva. The test strip may be a chemically treated cloth biomarker. The chemically treated cloth biomarker may be configured to identify bacteria in saliva that contacts the cloth test strip. The test strip may detect the level of bacteria present in the saliva and the level of bacteria may be determined based upon a color displayed on the test strip. The test strip may detect the level of bacteria present in the saliva and the level of bacteria may be determined based upon a color intensity displayed on the test strip.

The test strip may measure unbound hormones in the saliva, with the measured unbound hormones being tied to disease states. The test strip may measure lipidomic biomarkers. The test strip may measure key lipids, and the measured key lipids are used in diagnosing diseases, diagnosing disease states, and developing treatment plans. The test strip may be targeted to a specific disease state. The disease state may include one or more of diabetes, heart disease, autoimmune disorders, oral cancer, Alzheimer's, and gastrointestinal diseases, among other diseases. The test strip is designed to test oral health and/or extra-oral health.

The present disclosure provides a method that involves contacting one or more volatile organic compounds (VOCs) evolved from a sample to one or more color-changing detection regions configured for sensitivity against one or more VOCs, the contacting being performed such that the one or more color-changing regions attain a particular color profile following the contacting. The method also involves determining the presence or absence of one or more VOCs by comparing the particular color profile against a predetermined color profile map of color profiles corresponding to one or more VOCs.

The present disclosure also concerns a system that utilizes one or more color-changing detection regions configured such that the one or more color-changing regions attain a particular color profile following reaction with one or more VOCs evolved from a sample and evaluating the results to determine the existence of one or more VOCs. A predetermined color profile map of color profiles corresponding to one or more VOCs.

### BRIEF DESCRIPTION OF THE DRAWINGS

FIG. 1 depicts test strips for use in detecting oral health according to the invention according to the invention;

FIG. 2 depicts a front view of a breathalyzer/detector/analyzer according to the invention for testing breath and/or saliva;

FIG. 3 depicts an example process flow according to the invention; and

FIG. 4 depicts a system according to the invention.

### DETAILED DESCRIPTION

Oral health and hygiene are important for health. Poor oral health can result in disease to the body. Poor oral health can also be predictive of disease and overall health. The present invention is designed to test oral health to determine if a person needs to improve their oral hygiene and to assist in disease and general overall health diagnosis, among other things.

Both a breathalyzer and a sensor are described herein, as well as a method and system for testing oral health using a breathalyzer and/or sensor. The breathalyzer is a device that can capture droplets from the breath for testing purposes. The sensor is a strip or other device that is used to collect saliva, with the saliva directly contacting the strip and the strip having a colorimetric or other effect to signal to the user certain disease-related states. The strip may be placed directly in the mouth to collect saliva. Alternatively, the strip may be doused with saliva that is collected from the mouth, such as by spitting on the strip. The term breathalyzer and sensor are used here interchangeably, with the term breathalyzing meaning either a breathalyzer or a sensor and the term sensor meaning either a breathalyzer or a sensor.

Oral health is important in the context of orthodontia treatment. During treatment, patients are often challenged with oral hygiene. Food may get stuck between teeth and lodged in gums, causing swollen gums and excessive bacteria buildup. Patients are more likely to have bad breath. A breathalyzer 10 according to the invention provides a mechanism to measure the bacteria level in a patient's mouth and saliva to understand if a patient has bacteria present that may cause bad breath or that signals the existence of disease in the body. The breathalyzer 10 can determine which type of bacteria is present so that it can be properly treated.

The mouth, including mouth bacteria, is the gateway of these negative agents into the body. Saliva may be used to measure diagnostics amongst clinicians due to its measurable amount of free, unbound hormones that can be used to diagnose irregular hormone levels. In addition, hundreds of diverse types of bacteria live in the oral cavity, they are distinct to the palate, teeth, tongue, and tonsillar area.

Discerning these oral floras can illuminate bacterial infections that are present in the mouth and eventually continue into the human body. These can be connected to systemic infections, preliminary signs of bacterial endocarditis, pneumonia, cardiovascular disease, diabetes, gastrointestinal disorders, cancers and even Alzheimer's, among other disease states. The goal is to identify the exact bacteria or component that is related to each medical diagnosis of a systemic infection, precursor or symptom of a disease or disorder. Early diagnosis can be the difference between finding a solution and waiting for the symptoms to create a challenge. Thus, the present invention, as described herein, may serve as an early detection tool. The present invention connects the oral microbiota with the medical world and allows all specialists to be on the cutting edge of early detection and prevention.

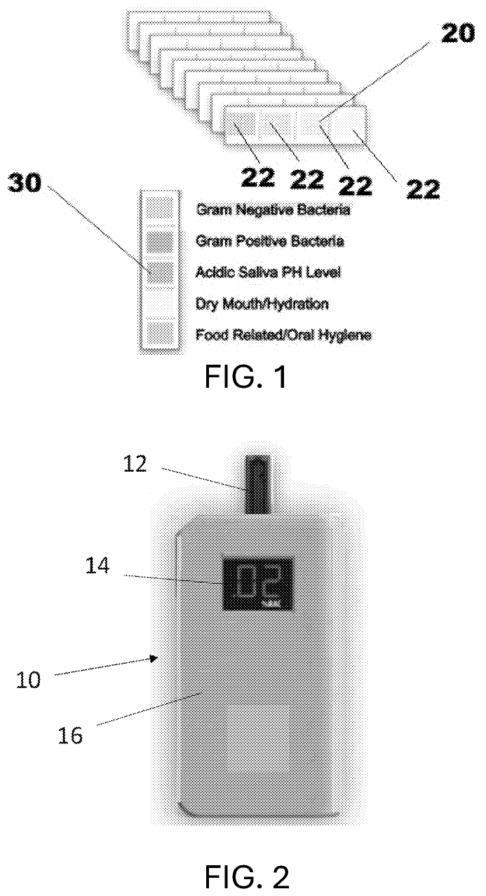

As shown in FIG. 1-2, a test strip 20 is provided. The test strip 20 may be used with a breathalyzer 10 or other testing device, or may be used by itself by placing the strip in the mouth or dipping the strip into a saliva sample. The test strip 20 is a small, pre-treated strip 20 that can measure the level of Ph, acidity, and bacteria, among other things, on the patient's tongue or in saliva. The test strip 20 may be considered a medical strip and is positioned on the tongue for approximately 10-15 seconds, until it is saturated, to allow the reaction. The test strip includes change detection regions 30, as shown in FIG. 4. The change detection regions 30 may show changes in color or may reveal symbols or other marks to indicate a positive reading. This is an easy-to-use tool that can show levels of bacteria and other levels of measured variables.

In one embodiment, the strip 20 can be a colorimetric sensor that depicts the levels of bacteria using color, such as shades of color. For example, red for high levels and light pink for low levels. In another embodiment, the color may be different colors, e.g., red for high levels and blue for low levels. Other signaling techniques may also be used, other than color. For example, a section of the test strip 20 could turn grey or another color to signal the level of the reading. Multiple different colors could be used on a single strip 20 to show varied and different types of bacteria, and/or other readable features of the microbiome, if desired. There could be multiple lines of testing on the same strip 20, or separate strips 20 could be used to measure different variables. For example, the test strip could have 5 or more testing lines for testing different agents. Alternatively, the test strip could have a 5×5 array of testing material sections. Instead of color, lines or symbols could appear in colored or uncolored backgrounds (similar to pregnancy tests).

The test strip 20 may be made of paper, plastic, cloth, or a combination thereof, or of other materials. The strip 20 may be coated with testing materials as known by those of skill in the art. Examples known in the art for colorimetric sensors, which are encompassed herein, include:
1) A change in light absorption of a chemochromic material
2) Nanocomposites employed as adsorbents. Examples of nanocomposites may include clay nanocomposites, hybrid nanocomposites, metal oxide-based nanomaterials, carbon nanotubes and various polymeric nanocomposites, which have been tested as adsorbents for the removal of a variety of heavy metals, such as arsenic, cadmium, cobalt, chromium, copper, mercury, manganese, nickel, lead, tin, and zinc from the aqueous medium. In addition to the utilization of nanocomposites alone, along with the materials such as graphene, single and multi-walled carbon nanotubes, and related hybrid materials.
3) Sensors that change in a physical or chemical environment when stimulated.
4) Schemes of chemical sensor design including receptor-spacer-reporter and indicator-displacement assay.
5) Optical nanostructures in which the refractive index changes periodically, such as photonic crystals.
6) Periodic dielectric, metallo-dielectric, superconductor microstructures or nanostructures that affect electromagnetic wave propagation.
7) Materials containing regularly repeating regions of high and low refractive index.
8) Materials including graphene and its derivatives, metal and metal oxide nanoparticles, DNA nanomaterials, quantum dots, and other materials.
9) Sensors based either on adsorption into a set of polymers or on oxidations at a set of heated metal oxides.
10) Tetrazolium dye and a carbon source.
11) Biochemical techniques, instrumental-based approaches, such as flow cytometry and gas chromatography, as well as spectroscopy-based techniques, such as Fourier transform infrared spectroscopy (FTIR) and Raman spectroscopy.
12) Silver-induced inhibition of urease activity and silver ion utilization by bacteria.
13) Dye with colorimetric and fluorescent properties.
14) Gold nanoparticles.
15) Fluorescent D Amino Acids.
16) Nanoporous pigment array constructed with composites of chemically responsive dyes and nanomaterials with expected properties for rapid and highly portable identification of trace VCs.
17) Photonic Crystals combined with dye, as disclosed in Nah, S. H., Kim, J. B., Chui, H. N. T., Suh, Y., & Yang, S., “Enhanced Colorimetric Detection of Volatile Organic Compounds Using a Dye-Incorporated Photonic Crystal-Based Sensor Array,” Advanced Materials, 36(46), 2409297 (2024), D. Kou, Y Zhang, S. Zhang, S. Wu, & W. Ma, “High-Sensitive and stable photonic crystal sensors for visual detection and discrimination of volatile aromatic hydrocarbon vapors,” Chem. Engr. J., Vol. 375, 121987 (Nov. 1, 2019), and L. Burratti, F. De Matteis, M. Casalboni, R. Francini, R. Pizzoferrato, & P. Prosposito, “Polystyrene photonic crystals as optical sensors for volatile organic compounds,” Materials Chemistry & Physics, Vol. 212, 274-281 (2018). Other articles also describe the relationship between VOC testing using photonic crystals and colorimetric sensing.

There are many different ways to detect VOCs known in the art that utilize a sensor strip on a substrate. The present invention is intended to encompass all known methods for detecting VOCs.

The testing materials preferably remain on the strip 20 during testing and are not left on a patient's tongue or in the patient's mouth. The testing materials could be flavored, in which case, the flavoring could be left on the patient's tongue. There may be additional ways for the strip 20 to measure beyond the mouth that involve collecting the saliva and testing it (e.g., patients could spit into small tubes or onto strips 20 instead of swiping the mouth with a strip 20). Saliva can also be transported through breath, such that small amounts of saliva can be collected from blowing into a device with a closed mouth.

The test strips 20 can be pretreated and then show a reaction, or the reaction can be brought on when the test strips 20 (pre-treated or not) are themselves treated with a solution or the like that causes a reaction. The reaction could be a color change, for example.

Referring to FIG. 2, the invention also includes a testing system for the mouth, such as a breathalyzer 10 that tests the type and level of bacteria in the mouth. This provides a means for eliminating bad breath and improving possible oral health issues and overall health issues. This system can be used for treating dry mouth, bad breath, gingival inflammation and other oral hygiene issues, including those associated with wearing braces. This system could also be used for detecting oral and extra oral disease states.

As shown in FIG. 2, the breathalyzer 10 may include a tab 12 at the top of the breathalyzer 10 that can be inserted into the mouth. The tab 12 may serve as a sensor for holding a test strip 20. Then the breathalyzer 10 reads the bacteria level or other disease states and may, for example, read out the level of bacteria on a scale, such as 1-5, where 5 is the worst. As shown in FIG. 6, the bacteria level may be presented on an electronic display 14. If the reading is at a higher level, this signals to the patient that they should use a bacteria killing product or agent. The testing device 10 may also automatically sense the types of VOCs and display the results automatically.

The breathalyzer 10 may be used at home and at the office to detect oral bacteria through saliva and unbound hormones, which can provide an early indication of potential health problems that can advance the standard of care as it relates to mouth care and healthcare. Lipid imbalance is closely associated with several diseases, such as atherosclerosis, diabetes, metabolic syndromes, systemic cancers, neurodegenerative diseases, and infectious diseases. Identification of lipidomic biomarkers or key lipids in different diseases can be used to diagnose diseases and disease states. The sensing device 12 can be used to test for both lipid imbalance and lipidomic biomarkers or key lipids, which can then be used for disease detection. In addition, they can be used to evaluate the response to treatments.

Biomarkers are objective, quantifiable characteristics of biological processes. The detector will initially detect bacteria that contributes to halitosis, including:
1. Gram negative bacteria located on the tongue and periodontal tissue;
2. Gram positive bacteria located on the enamel;
3. Bacteria located in saliva due to low PH or acidic saliva;
4. Bacteria production due to hydration and insufficient salivary flow; and
5. Bacteria due to food related/oral hygiene.

The sensor 12 may target 3-4 volatile organic compounds (“VOCs”) in saliva, such as ketones, alcohols, aldehyde and sulfur that are typically present in Staphylococcus aureus (S. aureus), one of the most common organisms associated with biofilms infections. Other pathogens associated with periodontitis are S. mutans, S. sanguis, P. gingivalis and F. nucleatum, which will have a different set of combination of ketones, alcohols, aldehyde, and sulfur of different compositions.

The sensor 12 and detector strip 20 are meant to allow for all medical facilities and lay persons to better understand the saliva and unbound hormones as it relates to underlying diseases. Early disease detection with saliva and its abundance of biomarkers is vital to reduce disease severity and prevent complications. As discussed above, the breathalyzer 10 can use a chemically treated test strip 20, such as a cloth or paper test strip 20, to solve for identifying the bacteria in the saliva that contributes to halitosis. The test strip 20 may have shapes other than an elongated rectangle. The test strips 20 can be easily placed into the breathalyzer 10 housing and removed therefrom. The breathalyzer 10 may alternatively include another means for capturing and testing saliva other than a test strip 20 that is removable and replaceable.

The same device can be used to detect different VOCs through the saliva and unbound hormones in the mouth. It may also be reusable, if desired.

The analyzer & detector read the type of salivary bacteria that is present in your mouth and links it to a specific disease state, on a scale differentiating different disease states. This can be done via an algorithm or computer program, if desired, and can be displayed on a computer or on an electronic display. This permits self-diagnosis similar to the scale with halitosis where you are predisposed or currently have the specific biomarkers for that disease state. This would be a tool similar to a breathalyzer 10 that you could also carry the strips 20, but it would be specific to each disease state below. This system can find 3 or 4 biomarkers for each disease state so that the user may become familiar with how it progresses and in what form. There can similarly be a chart to explain what the solution is based on the specific diagnosis from the lipidomic biomarker. The following disease states can be targeted; diabetes, heart disease, autoimmune disorders, oral cancers, Alzheimer's, and gastrointestinal diseases, among other disease states as discussed herein or otherwise known.

After using the breathalyzer 10, treatment options can be developed for use by the patient to resolve any problems identified. In addition, treatment options can be offered based upon results obtained from the test strip 20. For example, if the strip 20 turns bright red, signaling a high level of bacteria, the patient can be told to use a breath mint designed for reducing bacteria, or a mouth spray designed for reducing bacteria in the mouth. The mouth spray can be used to remove bacteria or to remove microbes, e.g., antibacterial spray or antimicrobial spray. The two features could be combined into a single spray such that one spray provides both antimicrobial and antibacterial properties. Anti-microbial treatments can be used to reduce or eliminate cavity production. Or it can be recommended that a patient use a tongue sponge.

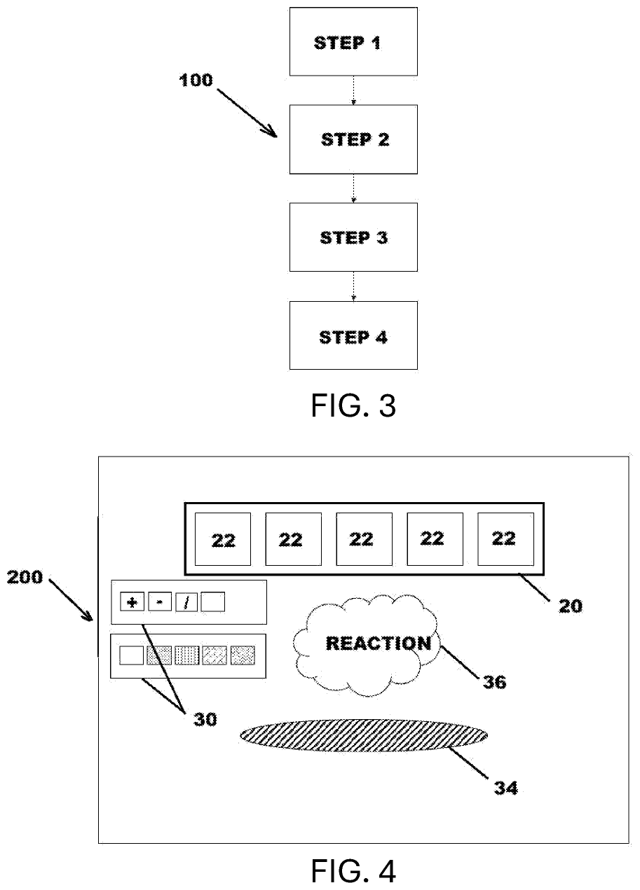

FIG. 3 depicts a flow chart 100 that identifies a process according to the invention. In step 1, a saliva or breath sample is collected. In step 2, VOC's evolve from the test sample. In step 3, a determination of the presence of a disease state can be made. In step 4, a diagnosis and/or treatment plan can be developed.

In step 1, saliva can be collected using any known technique, such as by swab, free-flowing saliva collection, spit collection, breath collection, or the like. The saliva can be collected on a test strip or transferred to a test strip.

In step 2, VOCs evolve from the sample, which can be accomplished by placing the saliva in a container and allowing VOC's evolved from the saliva to migrate into the headspace of the collection container. VOCs may be collected from a sensor that is put in the mouth so that saliva directly touches the sensor. The saliva may interact with the sensor to provide a change in color indicating the presence of VOCs, bacteria, or other chemicals in the saliva. It is preferred that the change depicted on the sensor strips be visible to the human eye. In an alternative embodiment, the test strip having a saliva sample may be placed into a reader device. If needed, the saliva sample can be subjected to, for example, heat, pressure, or other stimuli that encourages the release of VOCs from the saliva. It is preferred that an outside influence not be needed.

VOCs are contacted to one or more color changing detection regions that are configured for sensitivity against one or more VOCs on the testing strips. The VOCs to which the color-changing regions are sensitive can be, for example ketones, alcohols, aldehydes, sulfur, and the like. Exemplary color-changing regions are described in, for example, Nah, S. H., Kim, J. B., Chui, H. N. T., Suh, Y., & Yang, S., “Enhanced Colorimetric Detection of Volatile Organic Compounds Using a Dye-Incorporated Photonic Crystal-Based Sensor Array,” Advanced Materials, 36(46), 2409297 (2024). Color changing technology for detecting VOCs is also known from numerous other research articles, including, but not limited to, D. Kou, Y Zhang, S. Zhang, S. Wu, & W. Ma, “High-Sensitive and stable photonic crystal sensors for visual detection and discrimination of volatile aromatic hydrocarbon vapors,” Chem. Engr. J., Vol. 375, 121987 (Nov. 1, 2019), and L. Burratti, F. De Matteis, M. Casalboni, R. Francini, R. Pizzoferrato, & P. Prosposito, “Polystyrene photonic crystals as optical sensors for volatile organic compounds,” Materials Chemistry & Physics, Vol. 212, 274-281 (2018).

The VOCs can be characteristic of one or more bacteria of interest, which bacteria can be bacteria known to be associated with an oral or extraoral disease state. Following contact with the VOCs associated with the saliva sample, the color changing regions attain a particular color profile. The color profile may indicate the VOCs evolved from the saliva. The determination can be done in a manual or automated manner.

At step 3, it is determined whether VOCs are present are absent based upon the presence or absence of VOCs.

At step 4, a diagnosis or treatment plan can be provided based upon the detected oral or extra-oral disease state. For example, the patient can be told to brush better or more, or an antibiotic of steroid can be ordered.

FIG. 4 depicts a system according to the invention. The system 100 includes a sensor, such as a test strip 20, that utilizes change detection regions 22. The change detection regions 22 are configured to attain a change profile following a reaction 36 with a specified VOC that is evolved from a saliva sample 34. The change profile could be a color change, the revealing of a symbol, or other changes that signal to a user that a VOC is present. The sensor 20 could alternatively be a breath sensing device that utilizes a testing strip 20 that changes color when contacted with liquid particles from breath. The system may include a color profile map 30 that can be used to match the color change to a color profile in the map 30. The system 100 can be used to determine the presence or absence of an oral or extra-oral disease state based upon changes that occur on the test strip or sensor.

A separate reader (not shown) may be utilized to read the saliva sample or to receive the test strip/sensor. In one example, the color-changing regions reside within the system at distance from the test strip or other element that carries the saliva sample, and the VOCs evolved from the saliva sample then contact the color-changing regions.

The system may include one or more color-changing detection regions configured to have color changing regions that attain particular color region following a reaction with one or more VOCs evolved from the sample.

The system may include a color profile map that can be used as a reference for diagnosing the types of VOCs associated with the saliva sample using color. The color-changing regions may be present on a test strip.

In one embodiment, a method includes the steps of contacting one or more volatile organic compounds (“VOCs”) evolved from a sample to one or more detection regions and determining the presence or absence of a VOC. The determining of the presence or absence of a VOC is determined by comparing the change to the detection regions to a key, with the key being a visible change. The detection regions are configured for sensitivity against one or more VOCs. The contacting causes a change to the detection regions that is visible to the human eye.

The change to the detection region may be a change in color and the key may be a chart of colors, with the chart of colors being indicative of disease states. The change to the detection region may alternatively or in addition thereto be the revealing of a symbol and the key may be a chart of symbols.

The detection region may include photonic crystals associated with dye, with the photonic crystals changing color when contacted by the VOC. The detection region may be present on a test strip. The test strip may include multiple detection regions thereon.

The method may include inserting the test strip into a patient's mouth to collect a sample. The determining the presence or absence of a VOC of the method is performed utilizing collected images of the color-changing regions.

The sample may be a saliva sample. The method may also include determining the presence or absence of an oral or extra-oral disease state based upon the change found in the detection region of the test strip. The method may also include recommending or providing a treatment plan based on the presence of an oral or extra-oral disease state.

According to another embodiment, a system includes one or more detection regions configured to change based upon sensitivity against one or more VOCs when contacted with a respective VOC evolved from a sample and a key for analyzing the change in the detection region. The contacting of the one or more detection regions causes a change to the one or more detection regions that is visible to the human eye. The key is used to determine whether a disease state exists.

The change may be a color change and the key may be a predetermined color profile map of color profiles corresponding to one or more VOCs. The system may be configured to determine the presence or absence of a VOC by comparing the change in the one or more detection regions to the key.

The sample may be a saliva sample. The system may be further configured to determine the presence or absence of an oral or extra-oral disease state based on the presence of absence of the VOC. The change associated with the one or more detection regions may be a color change. The change associated with the one or more detection regions may be the revealing of a symbol in the one or more detection regions.

The one or more change detection regions may reside on a test strip. The system may optionally further include a reader configured to receive the test strip and to test the color against the key. The one or more change detection regions may reside on a test strip. The system may optionally further include a reader configured to receive the test strip and to test the symbol against the key.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim. The term “consisting essentially,” if used herein, means the specified materials or steps and those that do not materially affect the basic and novel characteristics of the material or method. The articles “a,” “an,” and “the,” should be interpreted to mean “one or more” unless the context indicates the contrary.

What is claimed is:

1. A method comprising:
contacting one or more volatile organic compounds (“VOCs”) evolved from a sample to one or more detection regions configured for sensitivity against one or more VOCs, with the contacting causing a change to the detection regions that is visible to the human eye; and
determining the presence or absence of a VOC by comparing the change to the detection regions to a key, with the key being a visible change.

2. The method of claim 1, wherein the change to the detection region is a change in color and the key is a chart of colors, with the chart of colors being indicative of disease states.

3. The method of claim 1, wherein the change to the detection region is the revealing of a symbol and the key is a chart of symbols.

4. The method of claim 2, wherein the detection region comprises photonic crystals associated with dye.

5. The method of claim 3, wherein the detection region comprises photonic crystals associated with dye.

6. The method of claim 1, wherein the detection region is present on a test strip.

7. The method of claim 6, wherein the test strip comprises multiple detection regions thereon.

8. The method of claim 6, wherein the test strip is inserted into a patient's mouth to collect a sample.

9. The method of claim 1, wherein the determining the presence or absence of a VOC is performed utilizing collected images of the color-changing regions.

10. The method of claim 8, wherein the sample is a saliva sample and further comprising determining the presence or absence of an oral or extra-oral disease state based upon the change found in the detection region of the test strip.

11. The method of claim 10, further comprising recommending or providing a treatment plan based on the presence of the oral or extra-oral disease state.

12. A system comprising:
one or more detection regions configured to change based upon sensitivity against one or more VOCs when contacted with a respective VOC evolved from a sample, with the contacting causing a change to the one or more detection regions that is visible to the human eye; and
a key for analyzing the change in the detection region to determine whether a disease state exists.

13. The system of claim 12, wherein the change is a color change and the key is a predetermined color profile map of color profiles corresponding to one or more VOCs.

14. The system of claim 12, wherein the system is configured to determine the presence or absence of a VOC by comparing the change in the detection region to the key.

15. The system of claim 12, wherein the sample is a saliva sample and wherein the system is further configured to determine the presence or absence of an oral or extra-oral disease state based on the presence of absence of the VOC.

16. The system of claim 12, wherein the change associated with the one or more detection regions is a color change.

17. The system of claim 12, wherein the change associated with the one or more detection regions is the revealing of a symbol in the one or more detection regions.

18. The system of claim 16, wherein the one or more change detection regions reside on a test strip, the system optionally further including a reader configured to receive the test strip and to test the color against the key.

19. The system of claim 17, wherein the one or more change detection regions reside on a test strip, the system optionally further including a reader configured to receive the test strip and to test the symbol against the key.
